SEQUENCE LISTING (4 sequences)

SEQ ID NO: 1 - DNA, 22 bp, Artificial Sequence
  Adaptor used for ligation
gcagaatgcgcggccgccttag

SEQ ID NO: 2 - DNA, 22 bp, Artificial Sequence
  Adaptor used for ligation
ctaaggcggccgcgcattctgc

SEQ ID NO: 3 - DNA, 44 bp, Artificial Sequence
  Circularization linker oligo
cgtcttacgcgccggcggaatccgtcttacgcgccggcggaatc

SEQ ID NO: 4 - DNA, 10 bp, Artificial Sequence
  Additional tail to the circulization linker; RCA primer
  misc_feature (10)..(10): Linked to six carbon linker and an amine group
aaaaaaaaaa

(19) United States
(12) Patent Application Publication
(10) Pub. No.: US 2007/0287151 A1
(43) Pub. Date: Dec. 13, 2007

(54) Methods and Means for Nucleic Acid Sequencing

(76) Inventor: Sten Linnarsson, Stockholm (SE)

Correspondence Address: COOLEY GODWARD KRONISH LLP;ATTN: Patent Group, Suite 1100, 777 - 6th Street, NW, WASHINGTON, DC, 20001, US

(21) Appl. No.: 10/593,785

(22) PCT Filed: Mar. 17, 2005
(86) PCT No.: PCT/EP05/02870

§ 371 Date: Jun. 13, 2007

Related U.S. Application Data
(60) Provisional application No. 60/555,954, filed on Mar. 25, 2004.

(30) Foreign Application Priority Data
Mar. 25, 2004 (GB) 0406769.0

(51) Int. Cl. C12Q 1/68; C07H 21/04; G01N 33/48; G06F 19/00

(52) U.S. Cl. 435/6; 536/23.1; 536/24.3; 702/20; 703/11

(57) ABSTRACT

Nucleic acid sequencing, especially high-density fingerprinting, in which a panel of nucleic acid probes is annealed to nucleic acid containing a template for which sequence information is desired, with determination of the presence or absence of sequence complementary to each probe within the template, thus providing sequence information. A reference sequence at least partly related to the template is used.

The present invention relates to nucleic acid sequencing.

The present invention especially relates to “high-density fingerprinting”, in which a panel of nucleic acid probes is annealed to nucleic acid containing a template for which sequence information is desired, with determination of the presence or absence of sequence complementary to each probe within the template, thus providing sequence information. The invention is based in part on using a reference sequence at least partly related to the template, overcoming various problems with existing sequencing techniques and allowing for a very large amount of sequence to be obtained in a single day using standard reagents and apparatus. Preferred embodiments allow additional advantages to be achieved. The invention also relates to algorithms and techniques for sequence analysis, and apparatus and systems for sequencing. The present invention allows for automation of a vast sequencing effort, using only standard bench-top equipment that is readily available in the art.

The invention involves hybridization of a panel of probes, each probe comprising one or more oligonucleotide molecules, in sequential steps determining for each probe if it hybridizes to the template or not, thus forming the ‘hybridization spectrum’ of the target. Preferably, the panel of probes and the length of the template strand are adjusted to ensure dense coverage of any given template strand with ‘indicative probes’ (probes which hybridize exactly once to the template strand). The invention further involves comparing the obtained hybridization spectrum with a reference database expected to contain one or more sequences similar to the template strand, determining the likely location or locations of the template strand within one or more reference sequences. The invention further allows for the hybridization spectrum of the template strand to be compared to the expected hybridization spectrum at the location or locations, thereby obtaining at least partial, sequence information of the template strand.

Although many different methods are used in genomic research, direct sequencing is by far the most valuable. In fact, if sequencing could be made efficient enough, then all three of the major scientific questions in genomics (sequence determination, genotyping, and gene expression analysis) could be addressed. A model species could be sequenced, individuals could be genotyped by whole-genome sequencing and RNA populations could be exhaustively analyzed by conversion to cDNA and sequencing (counting the number of copies of each mRNA directly).

Other examples of scientific and medical problems that can be addressed by sequencing include epigenomics (the study of methylated cytosines in the genome—by bisulfite conversion of unmethylated cytosine to uridine and then comparing the resulting sequence to an unconverted template sequence), protein-protein interactions (by sequencing hits obtained in a yeast two-hybrid experiment), protein-DNA interactions (by sequencing DNA fragments obtained after chromosome immunoprecipitation) and many other. Thus, highly efficient methods for DNA sequencing are desirable.

But in order to replace auxiliary methods such as microarrays and PCR fragment analysis, very high sequencing throughput is required. For example, a living cell contains about 300,000 copies of messenger RNA, each about 2,000 bases long on average. Thus to completely sequence the RNA in even a single cell, 600 million nucleotides must be probed. In a complex tissue composed of dozens of different cell types, the task becomes even more difficult as cell-type specific transcripts are further diluted. Gigabase daily throughput will be required to meet these demands. The table below shows some estimates on the throughput required for each experiment (humans, unless indicated otherwise):

| Experiment | Throughput required | |
|---|---|---|
| Genome sequence (10x de nova) | 30 | Gbp |
| Whole-genome polymorphisms | 3 | Gbp |
| Complete haplotype map (200 | 600 | Gbp |
| individuals) | | |
| Gene expression | 600 | Mbp |
| Epigenomics | 3 | Gbp |
| Ten million protein interactions | 400 | Mbp |
| Entire biosphere (one species per | ˜300 | Tbp |
| genus) | | |

The present invention place all of the above within reach at reasonable cost.

### BRIEF DESCRIPTION OF THE FIGURES

FIG. 1 shows a gel image which shows the result of cleaving a cDNA sample (lane 4) with CviJ* for increasingly long time. A gradual reduction in the average fragment length towards 100 bp is observed (100 bp is the lowest fragment of the size standard, lane 3). The optimal cleavage reaction is loaded in lane 1 and fragments around 100 bp are purified.

FIG. 2 shows adapter ligation. Lane 1 is the size marker, lane 2 unligated fragments, lanes 3 and 4 ligated fragments. Most fragments are correctly ligated.

FIG. 3 Shows the sample of fragments before (lane 1) and after (lane 2) circularization. Lane 3 shows the result after purification. Notice the absence of linker in lane 3.

FIG. 4 shows a section of approximately 0.8 by 2.4 mm from a random array slide scanned using a Tecan™ LS400 at 4 μm resolution using the 488 nm laser and 6FAM filter. Spots represent amplification products generated from individual circular template molecules.

FIG. 5 shows the stability of short oligonucleotide probes measured by melting point analysis:

FIG. 5A shows the effect of CTAB in 100 mM tris pH 8.0, 50 mM NaCl.

FIG. 5B shows the effect of LNA in TaqExpress buffer (GENETIX, UK).

FIG. 5C shows the specificity of LNA in TaqExpress buffer.

FIG. 5D shows the effect of introducing degenerate position: 7-mer with 5 LNA (left), 7-mer with 5 LNA and 2 degenerate positions (middle), 7-mer with 3 LNA and 2 degenerate positions (right).

FIG. 6 shows a FAM-labeled universal 20-mer probe (left panel) and a TAMRA-labeled 7-mer probe (middle), hybridized to a random array and visualized by fluorescence microscopy. The array was synthesized with two templates, both of which should bind the universal probe but only one of which should bind the 7-mer at the sequence CGAACCT. The image was captured using a Nikon DS1QM CCD camera at 20× magnification on a Nikon TE2000 inverted microscope. The right-hand panel shows a color composite, demonstrating that all TAMRA-labeled features were also FAM-positive, as expected.

### METHODS FOR DNA SEQUENCING

Sanger sequencing (Sanger et al. PNAS 74 no. 12: 5463-5467, 1977) using fluorescent dideoxy nucleotides is the most widely used method, and has been successfully automated in 96 and even 384-capillary sequencers. However, the method relies on the physical separation of a large number of fragments corresponding to each base position of the template and is thus not readily scalable to ultra-high throughput sequencing (the best current instruments generate ˜2 million nucleotides of sequence per day).

Sequences can also be obtained indirectly by probing a target polynucleotide with probes selected from a panel of probes.

Sequencing-by-hybridization (SBH) uses a panel of probes representing all possible sequences up to a certain length (i.e. a set of all k-mers, where k is limited by the number of probes that can fit on the microarray surface; with one million probes, k=10 can be used) and hybridizes the template. Reconstructing the template sequence from the set of probes is complicated and made more difficult by the inherently unpredictable nature of hybridization kinetics and the combinatorial explosion of the number of probes required to sequence larger templates. Even if these problems can be overcome, the throughput will necessarily be low, as one microarray carrying millions of probes is required for each template and the arrays are not usually reusable.

An alternative approach to SBH is to place the template on the solid surface and then sequentially hybridize the panel of probes. Using this approach, many templates can be sequenced in parallel, but the size of the panel of probes is necessarily limited by the sequential nature of the protocol. As a consequence, only very short templates can be sequenced. In fact the expected length that can be sequenced with k-mer probes is only 2^{k}, or 128 nucleotides using 16384 probes (k=7). With realistic hybridization times, such a protocol is not feasible. The authors of Drmanac et al. Nature Biotech 1998 (16):54-8) work around the problem by replicating each template on hundreds of separate membranes which can then be hybridized in parallel. However, such a workaround limits throughput and places additional demands on the template preparation method.

Nanopore sequencing (US Genomics, U.S. Pat. No. 6,355,420) uses the fact that as a long DNA molecule is forced through a nanopore separating two reaction chambers, bound probes can be detected as changes in the conductance between the chambers. By decorating DNA with a subset of all possible k-mers, it is possible to deduce a partial sequence. So far, no viable strategy has been proposed for obtaining a full sequence by the nanopore approach, although if it were possible, staggering throughput could in principle be achieved (on the order of one human genome in thirty minutes).

Various approaches have been designed for sequencing by synthesis (SBS).

In order to increase sequencing throughput it would be desirable to be able to visualize the incorporation of each base on a large number of templates in parallel, e.g. on a glass surface or similar reaction chamber. This is achieved by SBS (see e.g. Malamede et al. U.S. Pat. No. 4,863,849, Kumar U.S. Pat. No. 5,908,755). There are two approaches to SBS: either a byproduct released from each incorporated nucleotide is detected, or a permanently attached label is detected.

Pyrosequencing (e.g. WO9323564) determines the sequence of a template by detecting the byproduct of each incorporated monomer in the form of inorganic diphosphate (PPi). In order to keep the reactions of all template molecules synchronized, monomers are added one at a time and unincorporated monomers are degraded before the next addition. However, homopolymeric subsequences (runs of the same monomer) pose a problem as multiple incorporations cannot be prevented. Synchronization eventually breaks down (because lack of incorporation or misincorporation at a small fraction of the templates add up to eventually overwhelm the true signal), and the best current systems can read only about 20-30 bases with a combined throughput of about 200,000 bases/day.

While Sanger sequencing requires an elaborate apparatus (i.e. a capillary) for each template, Pyrosequencing is readily amenable to parallelization in a single reaction chamber. U.S. Pat. No. 6,274,320 describes the use of rolling-circle amplification to produce tandemly repeated linear single-stranded DNA molecules attached to an optic fiber, analyzed in a Pyrosequencing reaction which can then proceed in parallel. In principle, the throughput of such a system is limited only by the surface area (number of template molecules), the reaction speed and the imaging equipment (resolution). However, the need to prevent PPi from diffusing away from the detector before being converted to a detectable signal means that the number of reaction sites must be limited in practice. In U.S. Pat. No. 6,274,320, each reaction is constrained to occur in a miniature reaction vessel located on the tip of an optic fiber, thus limiting the number of sequences to one per fiber.

Even more limiting are the short read lengths achieved by Pyrosequencing (<50 bp). Such short sequences are not always useful in whole-genome sequencing, and the complex set of balancing reactions make it difficult to extend the read length much further. Only occasionally and for specific templates have read lengths up to 100 bp been reported.

A similar scheme with detection of a released label is described in U.S. Pat. No. 6,255,083. A scheme with sequential addition of nucleotides and detection of a label that is then cleaved off with an exonuclease is described in WO01/23610.

The principal advantage of detecting a released label or byproduct is that the template remains free of label at subsequent steps. However, because the signal diffuses away from the template, it may be difficult to parallellize such sequencing schemes on a solid surface such as a microarray.

The present invention in various aspects ingeniously addresses prior art problems.

The present invention in one aspect provide a sequencing method as set out in claim 1, with various embodiments as set out in dependent claims and within the description.

Within the method of claim 1, amplifying said template molecules by rolling-circle amplification may comprise adding polymerase and triphosphates under conditions which cause elongation of the amplification primer and strand displacement to form a tandem-repeated amplification product comprising multiple copies of the target sequence.

The panel of probes employed may be a full panel or a partial panel as explained further below.

The reference sequence for the sequence of the template will be a similar sequence. Similarity between a reference sequence and a template can be measured in many ways. For example, the proportion of identical nucleotide positions is commonly used. More advanced measures allow for insertions and deletions e.g. as in Smith-Waterman alignment and provide a probabilistic similarity score as in Durbin et al. “Biological Sequence Analysis” (Cambridge University Press 1998).

The degree of similarity required for the method of the present invention is determined by several factors, including the number and specificity of the probes used, the quality of the hybridization data, the template length and the size of the reference database. For example, simulations show that under the assumption of 5 degree melting point difference between match and mismatch probes (with 1 degree coefficient of variation), 256 probes and using the human genome as reference with 100 bp templates, then up to 5% sequence divergence can be tolerated. This corresponds for example to sequencing the Gorilla genome using the human genome as reference. Further increasing the number of probes, decreasing the length of the templates or improving the match/mismatch discrimination allows sequences of even lower similarity to be used as reference, e.g. 5-10%, up to 10%, 5-20%, 10-20% or up to 20%.

The present invention is applicable in various ways, including in resequencing, expression profiling, analysis or assessment of genetic variability, and epigenomics.

Nucleic acid to be sequenced may be any of interest, and may be or be obtained or derived from a whole genome, BACs, one or more chromosomes, cDNA and/or mRNA.

The input molecule or molecules may be for example be double-stranded or single-stranded, e.g. dsDNA, DNA/RNA, dsRNA, ssDNA or ssRNA.

Various embodiments may be performed as follows:

A first step (step 1) involves fragmentation, in particular creating a shotgun library of short fragments. Enzymatic and/or mechanical methods of generating fragments may be employed, for example including:
Enzymatic:
   Degradation with DnaseI (in the presence of Mn²⁺), then fill-in and/or enzymatic shortening of dangling ssDNA ends;
   Cutting with a moderately frequent cutter, such as MboI etc.;
   Partial cutting with a very frequent cutter, such as CviJI, CviJI* etc.;
   Cutting with a mix of restriction enzymes;
Mechanical:
   French press;
   Sonication;
   Shearing;
   each of which may be followed by enzymatic shortening and end-repair;
PCR
   using random priming sequences such as hexamers (optionally tailed with sequences for nested PCR);
   by PCR using degenerate primers or low-stringency conditions;
   by PCR using gene family-specific primers (etc.).

In the PCR approaches, this step may optionally be combined with step 2 by tailing the primers with sequence introducing an RCA (rolling circle amplification) primer annealing site.

Optionally following the first step a step “X” may be performed as described further below.

The second step (step 2) (which optionally follows step X) may involve introducing RCA primer annealing sequence. This may be for example by cloning into a vector (e.g. bacterial vector, phage etc.), then excising using restriction enzymes placed outside the cloning site as well as the primer motif; by ligation of double-stranded adaptors at one or both ends; or by ligation of hairpin adaptors at each end (causes simultaneous circularization). Optional additional, functional features that may be incorporated include features helping circularization and/or a helper oligo binding site, where a helper oligo can serve as donor or acceptor in FRET in downstream analyses.

Optionally following step 2 a step “X” may be performed as described further below.

A third step (step 3) may involve generating single-stranded circular DNA. This may be for example by ligation of hairpin adaptor after melting and self-annealing end-to-end in a maracas shape; by self-ligation of dsDNA followed by melting; by ligation to a helper fragment to form a dsDNA circle, followed by melting; by ligation of hairpin adaptors to both ends of dsDNA in a dumbbell shape; by self-ligation of ssDNA using helper linker (which may also serve as RCA primer).

Steps 2 and 3 may optionally be combined into a single step, for example in which circularization simultaneously introduces the RCA primer annealing sequence and any other desired features.

A fourth step (step 4) may involve rolling circle amplification (RCA). This may be in accordance with the following protocol:
Anneal an RCA primer to the circular ssDNA. The primer should carry a reactive moiety which can be used for immobilization.
Randomly immobilize the primer/template complex to the surface of an activated array using the attachment group of the RCA primer. The density of the primer/template complex on the surface should be optimized to allow for a maximum number of primer/template complexes on the surface without creating overlapping products after the RCA amplification (see below). The density of the primer/template complex on the surface may be controlled for example by the concentration of the primer/template complex, by the density of attachment sites on the surface and/or by the reaction conditions (time, buffer, temperature etc.).
Randomly immobilize the primer to the surface of an activated array using the attachment group of the RCA primer. The density of the primer on the surface should be optimized to allow for a maximum number of primer/template complexes on the surface without creating overlapping products after the RCA amplification (see below). The density of the primer on the surface may be controlled for example by the concentration of the primer, by the density of attachment sites on the surface and/or by the reaction conditions (time, buffer, temperature etc.).
Anneal an RCA primer to the circular ssDNA. The primer should carry a reactive moiety which can be used for immobilization.
After immobilisation and annealing: then
Add polymerase and the four dNTPs to initiate the rolling circle amplification.
Optionally incorporate fluorescent label in RCA which may serve as fluorescence donor or acceptor in FRET.
Optionally incorporate affinity tag in RCA which may be used for multiple purposes:
   For condensation of the RCA product by internal cross-linking using a multivalent linker molecule with affinity for the tag;
   For post-amplification labelling using a fluorescent label conjugated with a molecule with affinity for the tag;

Alternatively, RCA may be performed in solution and the product may be immobilized after amplification. For example, the same primer may be used for amplification and for immobilization. In another option, a modified dNTP carrying an immobilization group may be incorporated during amplification and the amplified product may then be immobilized using the incorporated immobilization group. For example, biotin-dUTP, or aminoallyl-dUTP (Sigma) may be used.

In a fifth step, step 5, sequence determination:
Determine the full or partial sequence of the various templates on the array using sequential hybridization of a panel of non-unique probes as described further below.
Optionally compare the sequence information for each template with a database of sequences representative of the sample under investigation thereby determining the relative proportion of each target within the sample and/or determining any genetic or other structural differences with respect to the database.

Step X has been mentioned already above. It is a step of selection of fragment size range (ideally with very good resolution—1-10% CV). Techniques that may be used include the following:
By gel electrophoresis and elution using
   PAGE with dsDNA
   PAGE with ssDNA
   Agarose gel;
By chromatography (e.g. HPLC, FPLC);
Using an affinity tag, e.g. a 3′-biotin on cDNA.

These steps provide disclosure of preferred and optional steps and ways of performing steps of a method in accordance with aspects and embodiments of the present invention. All combinations of disclosed features within the steps are provided herein as aspects and embodiments of the present invention as if set forth word-for-word herein.

The present invention is based on development of a novel sequencing strategy that improves on previously described sequencing methods while allowing for most of their difficulties to be avoided. It is a strategy that is easy to parallelize (no size fractionation is required) and that provides the possibility for long read lengths.

A method in accordance with the present invention may comprise three fundamental steps. First, a random array of locally amplified template molecules is generated (preferably in a single step) from a sample containing a plurality of template strands. Second, the random array is subjected to sequential hybridization with a panel of probes with determination of the presence or absence of sequences complementary to each probe in each amplified template on the array. Third, the hybridization spectrum thus obtained is compared to a reference sequence database with a method that allows the determination of likely insertions, deletions, polymorphisms, splice variants or other sequence features of interest. The comparison step may be further separated in a search step followed by an alignment step.

Random Array Synthesis

There are many approaches to providing amplified templates at high density. First, amplified templates may be arrayed by mechanical means, which however requires separate amplification reactions for each individual template molecule (thus limiting throughput and increasing cost). Second, templates may be amplified in situ using in-gel PCR (e.g. as described in U.S. Pat. No. 6,485,944 and Mitra R D, Church G M, “In situ localized amplification and contact replication of many individual DNA molecules”, Nucleic Acids Research 1999: 27(24):e34), which however requires the use of a gel (thus severely interfering with subsequent hybridization reactions).

The present invention advantageously uses rolling-circle amplification to synthesize random arrays in a single reaction from a sample containing a plurality of template molecules. Densities up to 10⁵-10⁷ per mm² are achievable. A random array synthesis protocol employed in embodiments of the present invention may comprise:

a. Provide a surface (e.g. glass) with an activated surface.

b. Attach primers, preferably via a covalent bond, or, instead of a covalent bond, a strong non-covalent bond (such as biotin/streptavidin) may be used.

b. Add circular single-stranded templates, preferably at a density suitable for the detection equipment.

c. Anneal the templates to the primers.

d. Amplify using rolling-circle amplification to produce a long single-stranded tandem-repeated template attached to the surface at each position.

Lizardi et al. describe “Mutation detection and single-molecule counting using isothermal rolling circle amplification”: Nature Genetics vol 19, p. 225.

Modifications to this procedure include preannealing the circular template molecules to activated primers before immobilization, and/or providing “open-circle” template molecules which are circularized upon annealing to the primer and closed using a ligation reaction.

A “suitable density” is preferably one that maximizes throughput, e.g. a limiting dilution that ensures that as many as possible of the detectors (or pixels in a detector) detect a single template molecule. On any regular array, a perfect limiting dilution will make 37% of all positions hold a single template (because of the form of the Poisson distribution); the rest will hold none or more than one.

For example, on a Tecan LS400 with a 6 μm pixel size, the 7.5×2.2 cm reaction surface holds 45 million pixels. With a limiting dilution (Poisson distribution), 37% of those would hold a single template, i.e. 17 million templates. Sequencing 150 nucleotides on each template yields 2.5 Gb of sequence in 150 cycles. With a cycle time of 5 minutes, daily throughput is about 5 Gbp, equivalent to two full sequences of the human genome. In practice, more than one pixel may be needed to reliably detect a feature, but the same reasoning holds whether the detector is a single pixel or multiple pixels.

Templates suitable for solid-phase RCA should optimize the yield (in terms of number of copies of the template sequence) while providing sequences appropriate for downstream applications. In general, small templates are preferable. In particular, templates can consist of a 20-25 bp primer binding sequence and a 40-500 bp insert, which may be a 40-150 bp insert. However, templates up to 500 bp or up to 1000 bp or up to 5000 bp are also possible, but will yield lower copy numbers and hence lower signals in the sequencing stage. The primer binding sequence may be used both to circularize an initially linear template and to initiate RCA after circularization, or the template may contain a separate RCA primer binding site.

In order to increase the signal generated from rolling-circle amplified templates it may be necessary to condense them. Since an RCA product is essentially a single-stranded DNA molecule consisting of as many as 1000 or even 10000 tandem replicas of the original circular template, the molecule will be very long. For example, a 100 bp template amplified 1000 times using RCA would be on the order of 30 μm, and would thus spread its signal across several different pixels (assuming 5 μm pixel resolution). Using lower-resolution instruments may not be helpful, since the thin ssDNA product occupies only a very small portion of the area of a 30 μm pixel and may therefore not be detectable. Thus, it is desirable to be able to condense the signal into a smaller area.

In (Lizardi et al, cited above) the RCA product is condensed by using epitope-labeled nucleotides and a multivalent antibody as crosslinker. Alternative approaches include biotinylated nucleotides cross-linked by streptavidin.

Alternatively, condensation may be achieved using DNA condensing agents such as CTAB (see e.g. Bloomfeld ‘DNA condensation by nultivalent cations’ in ‘Biopolymers: Nucleic Acid Sciences’).

In order to immobilise the RCA primer oligonucleotides to a surface, many different approaches have been described (see e.g., Lindroos et al. “Minisequencing on oligonucleotide arrays: comparison of immobilisation chemistries”, Nucleic Acids Research 2001: 29(13) e69). For example, biotinylated oligos may be attached to streptavidin-coated arrays; NH₂-modified oligos may be covalently attached to epoxy silane-derivatized or isothiocyanate-coated glass slides, succinylated oligos may be coupled to aminophenyl- or aminopropyl-derived glass by peptide bonds, and disulfide-modified oligos may be immobilised on mercaptosilanised glass by a thiol/disulfide exchange reaction. Many more have been described in the literature.

Resequencing by Sequential Hybridization of Short Probes

The sequencing approach of the present invention comprises hybridization of a panel of probes, with match/mismatch discrimination for each probe and target. The result is a “spectrum” of each target. Furthermore, a reference sequence is provided in which the spectrum is located and aligned so that differences in the sequence of the target with respect to the reference can be determined with high accuracy.

The panel of probes and the target length are optimized so that the spectra can be used both (1) to locate unambiguously each target sequence in the reference sequence and (2) to resolve accurately any sequence difference between the target and the reference sequence.

In order to fulfill the first requirement, the panel contains enough information (in the information-theoretic sense) to unambiguously locate the target. A single, long, specific probe is sufficient to locate a single specific target, but cannot be used since that would require separate probes for each possible target. Instead, short non-unique probes are used. An optimal panel would use probes with a 50% statistical probability of hybridizing to each target, corresponding to 1 bit of information per probe. 50 such probes would be capable of discriminating more that 1000 billion targets. Such panels have the additional advantage of being resilient to error and to genetic polymorphisms. Our experiments have shown that a panel of 100 4-mer probes is capable of uniquely placing 100 bp targets in the human transcriptome even in the presence of up to 10 SNPs.

In order to fulfill the second requirement, the panel of probes must cover the target and must be designed such that sequence differences result in unambiguous changes in the spectrum. For example, a panel of all possible 4-mer probes would completely cover any given target with four-fold redundancy. Any single-nucleotide change would result in the loss of hybridization of four probes and the gain of four other characteristic probes.

The sensitivity of a probe panel can be calculated:

A probe is a mixture of one or more oligonucleotides. The mixture and the sequence of each oligonucleotide defines the specificity of the probe. The dilution factor of a probe is the number of oligonucleotides it contains. The effective specificity of a probe is given by the length of a non-degenerate oligonucleotide with the same probability of binding to a target. For example, a 6-mer probe consisting of four oligonucleotides where the first position is varied among all four nucleotides (i.e. is completely degenerate) has an effective specificity of 5 nucleotides.

A panel is a set of k-mer probes with the property that any given k long target is hybridized by one and only one probe in the panel. Thus, a panel is a complete and non-redundant set of probes.

The complexity C of a probe panel is the number of probes in the panel.

The sensitivity of a position within a panel is the set of different targets it can discriminate at that position. For example, a panel where the probes are either GC mixed or AT mixed at a position (denoted GC/AT) is sensitive to G-A, C-A, C-T and G-T differences (i.e. transitions), but not to transversions (G to C etc).

When probing with a full panel of probes, each position in the target is guaranteed to be probed by each position in the panel, i.e. by k staggered overlapping probes. However, the sensitivity of each position may be different, so that some differences in the target are only detectable by less than k probes.

For example, the panel given by (GCAT) (GC/AT) (GC/AT) (G/C/A/T) (G/C/A/T) (GC/AT) (GC/AT) (GCAT) has 8 positions (i.e. k=8). The first and last position are completely degenerate, so no change in the target is detected by those positions. Transitions (GC<->AT) are detected by 6 positions, while transversions (GA<->CT) are detected by only two positions in each probe. The effective specificity can be calculated by summing the effective specificity of each position: 0+0.5+0.5+1+1+0.5+0.5+0=4 bp.

For non-trivial targets, it will often be the case that probes are repeated in the target. Such probes lose their sensitivity to changes at any single position, since they will still hybridize to the other.

Given the length L of the target, we can calculate the probability (for each position in the target) that there is at least one probe sensitive to a change at that position.

First, we need to find out how many probes are sensitive to the change of interest in a repeat-free target. Call this k_{c}; k_{c} is 6 for transitions and 2 for transversions in the previous example.

Then, we note that the probability p(R) that any given probe is present in one or more of the other positions in the target (i.e. that it is repeated) is

$$P ( R ) = 1 - {( \frac{C - 1}{C} )}^{L - 1}$$

The probability p(S) that not all of the 2k_{c} sensitive probes are repeated is then
P(S)=1−P(R)^{2k}^{ₑ}

The exponent is 2k_{c} because any change causes the disappearance of k_{c} probes and the appearance of k_{c} new probes.

We can now calculate the sensitivity given the target length. For example, C=256, k_{c}=2, L=120 gives p=98%, i.e. the panel with 256 probes is sensitive to 98% of all transversions (and 100% of transitions, k_{c}=6). If we use only half of the probes in the panel, so that the effective k_{c}=1, then p=86% for transversions and 99.7% for transitions (k_{c}=3). The overall average sensitivity in a species like the human (which has 63% transitions) would be 95%.

The theory is strictly valid as long as the number of SNPs is low compared with the target length—i.e. as long as multiple SNPs do not occur within the length one probe. In practical experiments this is almost always true: for example, human genomic DNA contains about 1 SNP per 1000 nucleotides, and two SNPs within 7 bases is thus very unlikely.

In practice, we may require at least two sensitive probes to score a SNP (i.e. because hybridization data is error-prone). In that case, the probability P(S) becomes 1−p(R)^{2kc-1} and the calculations are again straightforward.

When working with subsets of panels (in order to save time and reagents), it may desirable to nevertheless guarantee that any position in the target is probed on one strand or the other. In other words, we seek a subset of probes such that any k-mer which is not probed is guaranteed to be probed on the opposite strand. Such subsets can be obtained by placing (G/A), (C/T), (G/T) or (C/A) in the middle position. For example (G/A) will fail to probe G and A in the target, in which case the opposite strand is guaranteed to be either C or T, which are probed. Other variations are possible.

The (GC/AT) degenerate position has two desirable features. First, it guarantees that the individual oligos in each probe have similar melting point (since they will either be all GC or all AT). Second, the position will be sensitive to transitions which represent 63% of all SNPs in humans.

Hybridization of Short Oligomer Probes

In the present invention, it is envisaged that a panel of probes is sequentially hybridized to the targets. In order to limit the complexity of the panel of probes, it is desirable to keep the probes short, preferably to have only 3-6 bp effective specificity. Here we describe the requirements for hybridizing short oligomer probes.

The probes are stabilized in order for them to hybridize effectively, or at all. In addition, stabilization may help the probe compete with any internal secondary structure that may be present in the target. Stabilization can be achieved in many different ways.
Through stabilizing additives in the hybridization reaction, for instance salt, CTAB, magnesium, stabilizing proteins.
Through the addition of degenerate positions that extend the length of the probe without increasing its complexity. For example, a 6-mer probe extended with an ‘N’ position would really be a mixture of four oligonucleotides, each 7 bases long. A (GC/AT) position—indicating a mix of G and C or a mix of A and T—would extend the probe by one base while only doubling the complexity (instead of quadrupling it).
Through modification of the probe chemistry, for example by means of locked nucleic acid (Exiqon, Denmark), peptide nucleic acid and or minor groove binder (Epoch Biosciences, US).
A combination of the above, for example a degenerate probe with LNA hybridized in CTAB buffer.

Of these, the first will also stabilize the target (thus potentially inducing stable secondary structures which prevent hybridization). Methods that stabilize the probe selectively are preferred.

Detecting Hybridization

Many approaches are known for detecting hybridization.
Direct fluorescence. The probe is labeled and hybridization is detected by the increased local concentration of probes hybridized to the target. This may require high magnification, confocal optics or total internal reflection excitation (TIRF).
Energy transfer. The probe is labeled with a quencher or donor and the target is labeled with counterpart donor or quencher. Hybridization is detected by the decrease of donor fluorescence and/or the increase in quencher fluorescence.
Single-base extension. The hybridized probe serves as primer for a single base extension reaction incorporating fluorescent dye (alternatively, released PPi maybe detected as in Pyrosequencing).

A preferred approach is described:

The probe is labeled by a fluorophor detectable in an epifluorescence microscope or a laser scanner, for example Cy3. Many other suitable dyes are commercially available. The probe is hybridized to the array at a concentration optimized to permit detection of the local increase in concentration at a hybridized array feature, over the background present in all the liquid. For example, 400 nM may be used, or the probe may be hybridized at 1 nM up to 500 nM or even 500 nM up to 5 μM depending on the optical setup. The advantage of this detection scheme is that it avoids a washing step, so that detection can proceed at equilibrium hybridization conditions, which facilitates match/mismatch discrimination.

An energy transfer approach is described:

The target carries a permanently hybridized helper oligonucleotide with a fluorescence donor. The helper is designed to withstand washes that would melt away the short probes. The probes carry a dark quencher. For example, the donor may be fluorescein and the quencher Eclipse Dark Quencher (Epoch Biosciences). Many other donor/quencher pairs are known (see e.g. Haugland, R. P., ‘Handbook of fluorescent probes and research chemicals’, Molecular Probes Inc., USA). In general, it is desirable to have a probe with a long Förster radius, capable of quenching over long distances. Hybridization is detected by the quenching of the donor fluorophor upon hybridization of the probe.

Spectral Search and Alignment

Given the spectrum of a target, we first seek the location of the target within the reference sequence, allowing for sequence differences. The search can be performed by simply scanning the reference sequence with a window of the same size as the target, computing an expected spectrum for each position and comparing the expected spectrum with the observed spectrum at the position. The highest-scoring position or positions are returned.

Because the method of the invention generates very large numbers of hybridization spectra in a short time, it is important to optimize the search step. For example, in a current implementation, spectral search proceeds at 1.2 billion matches per second on a high-end workstation, and we estimate that ten workstations will be required to keep up with a single sequencing instrument. It is another aspect of the invention to accelerate the search using programmable hardware, i.e. field-programmable gate arrays (FPGA). By translating the search algorithm to Mitrion-C (Mitrion AB, Sweden), an acceleration of 30 times can be achieved using just two FPGA chips in a single workstation computer.

Once one or more likely locations have been found, we seek a modification to the reference sequence that will explain any discrepancies between the observed and expected spectra. We may at this stage introduce relevant modifications to the reference sequence, e.g. SNPs, short indels, long indels, microsatellites, splice variants etc. For each modification or combination of modifications, we again compute a score for the similarity between the observed and expected spectra. The most likely modified reference sequence or sequences are returned. Methods for searching very large parameter spaces are known in the art, e.g. Gibbs sampling, Markov-chain Monte Carlo (MCMC) and the Metropolis-Hastings algorithm.

When comparing spectra, a simple binary overlap score may be used (scoring 1 for each probe that either does or does not hybridize in both spectra, 0 otherwise), or a more sophisticated statistical approach may use gradual or probabilistic measures of spectral overlap.

Where multiple targets locate to the same position in the target, higher-level analysis may then be performed to assess the confidence in any sequence differences.

An Apparatus for Automated High-Throughput Sequencing

Methods according to the present invention are particularly suitable for automation, since they can be performed simply by cycling a number of reagent solutions through a reaction chamber placed on or in a detector, optionally with thermal control.

In one example, the detector is a CCD imager, which may for example be operating by white light directed through a filter cube to create separate excitation and emission light paths suitable for a fluorophore bound to each target. For instance, a Kodak KAF-16801E CCD may be used; it has 16.7 million pixels, and an imaging time of ˜2 seconds. Daily sequencing throughput on such an instrument would be up to 10 Gbp.

The reaction chamber provides:
easy access for the optics.
a closed reaction chamber.
an inlet for injecting and removing reagents from the reaction chamber.
an outlet to allow air and reagents to enter and exit the chamber.

A reaction chamber may be constructed in standard microarray slide format as shown in FIG. 3, suitable for being inserted in an imaging instrument. The reaction chamber can be inserted into the instrument and remain there during the entire sequencing reaction. A pump and reagent flasks supply reagents according to a fixed protocol and a computer controls both the pump and the scanner, alternating between reaction and scanning. Optionally, the reaction chamber may be temperature-controlled. Also optionally, the reaction chamber may be placed on a positioning stage to permit imaging of multiple locations on the chamber.

A dispenser unit may be connected to a motorized valve to direct the flow of reagents, the whole system being run under the control of a computer. An integrated system would consist of the scanner, the dispenser, the valves and reservoirs and the controlling computer.

In accordance with a further aspect of the invention there is provided an instrument for performing a method of the invention, the instrument comprising:
an imaging component able to detect an incorporated or released label,
a reaction chamber for holding one or more attached templates such that they are accessible to the imaging component at least once per cycle,
a reagent distribution system for providing reagents to the reaction chamber.

The reaction chamber may provide, and the imaging component may be able to resolve, attached templates at a density of at least 100/cm², optionally at least 1000/cm², at least 10 000/cm² or at least 100 000/cm², or at least 1 000 000/cm², at least 10 000 000/cm² or at least 100 000 000 per cm².

The imaging component may for example employ a system or device selected from the group consisting of photomultiplier tubes, photodiodes, charge-coupled devices, CMOS imaging chips, near-field scanning microscopes, far-field confocal microscopes, wide-field epi-illumination microscopes and total internal reflection miscroscopes.

The imaging component may detect fluorescent labels.

The imaging component may detect laser-induced fluorescence.

In one embodiment of an instrument according to the present invention, the reaction chamber is a closed structure comprising a transparent surface, a lid, and ports for attaching the reaction chamber to the reagent distribution system, the transparent surface holds template molecules on its inner surface and the imaging component is able to image through the transparent surface.

A further aspect of the invention provides a random array of single-stranded DNA molecules, wherein
each said molecule consists of at least two tandem-repeated copies of an initial sequence,
each said molecule is immobilized on a surface at random locations with a density of a density of between 10³ and 10⁷ per cm², preferably between 10⁴ and 10⁵ per cm², or preferably between 10⁵ per cm² and 10⁷ per cm²,
each said initial sequence represents a random fragment from an initial target DNA or RNA-library comprising a mixture of single- or double-stranded RNA or DNA molecules,
said initial sequences of all said DNA molecules have approximately the same length.

Generally, the molecules will comprise at least 100 tandem-repeated copies of an initial sequence, usually at least 1000, or at least 2000, preferably up to 20 000. The molecules may comprise 50 or more tandem-repeated copies of an initial sequence, which is detectable using standard microscopy.

Preferably, the initial sequences have the same length within 50% CV, preferably 5-50% CV, preferably within 10% CV, preferably within 5% CV i.e. such that the distribution is such that the coefficent of variation (CV) is e.g. 5%. CV=standard deviation divided by the mean. The initial sequences may have the same length.

The initial target library may for example be or comprise one or more of an RNA library, an mRNA library, a cDNA library, a genomic DNA library, a plasmid DNA library or a library of DNA molecules.

A further aspect of the invention provides a set or panel of probes wherein
each probe consists of one or more oligonucleotides,
each said oligonucleotide is stabilized,
each said oligonucleotide carries a reporter moiety,
the effective specificity of each probe is between 3 and 10 bp,
the set of probes statistically hybridizes to at least 10% of all positions in a target sequence.

The effective specificity may be between 4 and 6 bp. The effective specificity may be 3, 4, 5, 6, 7 8, 9 or 10 bp.

The set of probes may statistically hybridize to at least 25%, at least 50%, at least 90% of all positions in a target sequence, or to 100% of all positions in a target sequence.

The set of probes may hybridize to 100% of all positions in a target sequence or its reverse complement, such that each position in the target or the reverse complement of the target at that position is hybridized by at least one probe in the set.

The target sequence may be an arbitrary target sequence.

A set of probes according to the invention may be stabilised by one or more of introduction of degenerate positions, introduction of locked nucleic acid monomers, introduction of peptide nucleic acid monomers and introduction of a minor groove binder.

The reporter moiety may for example be selected from the group consisting of a fluorophor, a quencher, a dark quencher, a redox label, and a chemically reactive group which can be labeled by enzymatic or chemical means, for example a free 3′-OH for primer extension with labeled nucleotides or an amine for chemical labeling after hybridization.

Examples of Applications

Gene Expression Profiling

By sequencing cDNA fragments at random, the expression level of the corresponding RNA can be quantified by counting the number of occurrences of fragments from each RNA. Structural features (splice variants, 5′/3′ UTR variants etc.) and genetic polymorphisms can be simultaneously discovered.

Genetic Profiling

Shotgun sequencing of whole genomes can be used to genotype individuals by noticing the occurrence of sequence differences with respect to the reference genome. For example, SNPs and indels (insertion/deletion) can easily be discovered and genotyped in this way. In order to discriminate heterozygotic sites, dense fragment coverage may be required to ensure that both alleles will be sequenced.

Further aspects and embodiments of the present invention will be apparent to the skilled person in the light of the present disclosure. All documents cited anywhere in the specification are incorporated by reference.

### EXAMPLE 1

### Preparing DNA Templates for Cantaloupe

Input

Double stranded DNA template.

Template Fractionation:

We used the restriction enzyme CviJ I* (EURx, Poland), that recognizes 5′-GC-3′ and cuts blunt in between. We set up restriction reactions as follows:

| | | |
|---|---|---|
| 1 ug Template | 1.5 ug Template | 2 ug Template |
| 2x reaction buffer | 2x reaction buffer | 2x reaction buffer |
| 25 ul | 25 ul | 25 ul |
| 0.3 units CviJ I* | 0.3 units CviJ I* | 0.3 units CviJ I* |
| Water to 50 ul | Water to 50 ul | Water to 50 ul |
| Total volume | Total volume | Total volume |
| 50 ul | 50 ul | 50 ul |

Reactions were incubated for 1 hour at 37° C.

The cleaved DNA was purified with PCR cleanup kit (Qiagen) according to manufacturer's protocol.

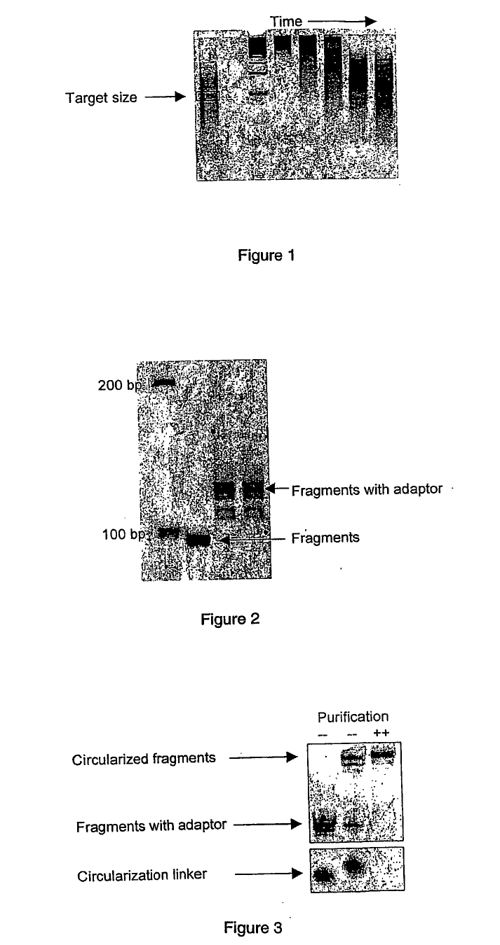

We analyzed a fraction on a 2% agarose gel and identified the optimal reaction conditions for the specific batch of template and enzyme (see FIG. 1, lanes 4-8).

We repeated the optimal cleavage reaction to get a total of 5 ug DNA (FIG. 1, lane 1).

Template Size Selection:

We purified the DNA on an 8% non denaturing PAGE (40 cm high, 1 mm thick). Each well was loaded with no more than 1 μg of DNA, and a 95-105 ladder was included, indicating the region of interest. The ladder consisted of 3 PCR fragments, at 95, 100 and 105 base pairs.

We stained the gel with SYBR gold and analyzed the result on a scanner, cut out the region of interest (95-105 bp) and electro-eluted the desired range of DNA with ElutaTube™ (Fermentas) according to manufactures protocol.

Adaptor Ligation:

One adaptor was used for ligation.

5′ GCAGAATGCGCGGCCGCCTTAG 3′
3′ CGTCTTACGCGCCGGCGGAATC 5′

It contained 5′ phosphates and an internal Not I site.

We prepared the following ligation mixture

1 pmol of DNA (60-70 ng of
fractionated sample)
25 pmol adaptor
Quick ligation buffer (NEB)
20 ul
Water up to 40 ul
Quick ligase (NEB)
2 ul
Total volume
42 ul

Incubated at 25° C. for 15 minutes.

Purified using PCR cleanup (Qiagen) according to manufacturer's protocol. See FIG. 2.

Restriction Digest Not I:

We set up the following reaction:

Ligated DNA (all of it)
10x buffer (NEB)
10 ul
100x BSA
1 ul
Water up to 95 ul
Not I (50 units)
5 ul

Incubated at 37° C. for 4 hours or overnight.

Purified sample using PCR cleanup (Qiagen) according to manufactures protocol.

We repeated the purification with PCR cleanup to remove as much as possible of excess adaptors.

Circularization of Templates:

We formed single stranded circles by denaturing the samples in the presence of linker oligo

5′-CGTCTTACGCGCCGGCGGAATCCGTCTTACGCGCCGGCGGAAT
C-3′.

We mixed the following

Ligated and Not I cut
sample (everything)
5 pmol of linker oligo
Water up to 50 ul

Heated to 93° C. for 3 minutes, put on ice until cold, quick spin.

Added 50 ul of 2× Quick ligation buffer (NEB) and 1 ul of Quick ligase (NEB), mixed briefly.

Incubated 25° C. for 15 minutes.

At this stage the circles are formed and the samples can go on for RCA. See FIG. 3.

Immobilization:

5 μM RCA primer (identical to the circularization linker with an additional 5′-AAAAAAAAAA-C6-NH-3′ tail, where C6 is a six-carbon linker and NH is an amine group) was immobilized on SAL-1 slides (Asper Biotech, Estonia) in 100 mM carbonate buffer pH 9.0 with 15% DMSO.

Incubated at 23° C. for 10 hours.

Remaining active sites on the slide surface were blocked by first soaking in 15 mM glutamic acid in carbonate buffer (as above, but 40 mM) at 30° C. for 40 minutes, then soaking in 2 mg/ml polyacrylic acid, pH 8.0 in room temperature for 10 minutes.

Circular templates were annealed at 30° C. in buffer 1 (2×SSC, 0.1% SDS) for 2 hours, then washed in buffer 1 for 20 minutes, then washed in buffer 2 (2×SSC, 0.1% Tween) for 30 minutes, then rinsed in 0.1×SSC, then rinsed in 1.5 mM MgCl₂.

Amplification:

Rolling-circle amplification was performed for 2 hours in Phi29 buffer, 1 mM dNTP, 0.05 mg/mL BSA and 0.16 u/μL Phi29 enzyme (all from NEB, USA) at 30° C.

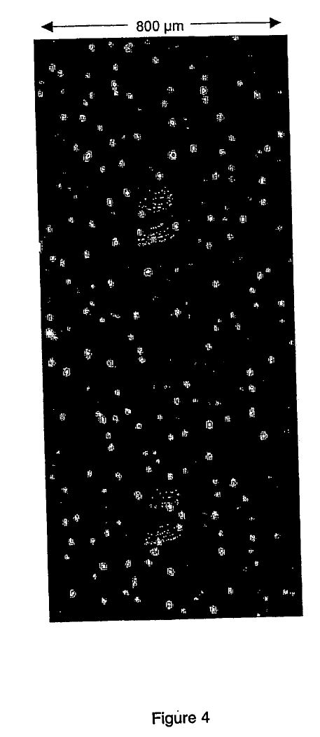

Reporter oligonucleotide complementary to the circularization linker and labelled with 6-FAM was annealed as above, followed by soaking in buffer 3 (5 mM Tris pH 8.0, 3.5 mM MgCl₂, 1.5 mM (NH₄)₂SO₄, 0.01 mM CTAB). FIG. 4 shows a small portion of a slide with individual RCA products clearly visible.

Probe Panel Hybridization:

Each probe was designed according to the following scheme: (GCAT) (GC/AT) (GC/AT) (G/C/A/T) (GC/AT) (G/C/A/T) (GC/AT), each with locked nucleic acid (Exiqon, Denmark) at positions 2, 4 and 6 and with Eclipse dark quencher (Epoch Biosciences, USA) at the 3′ end.

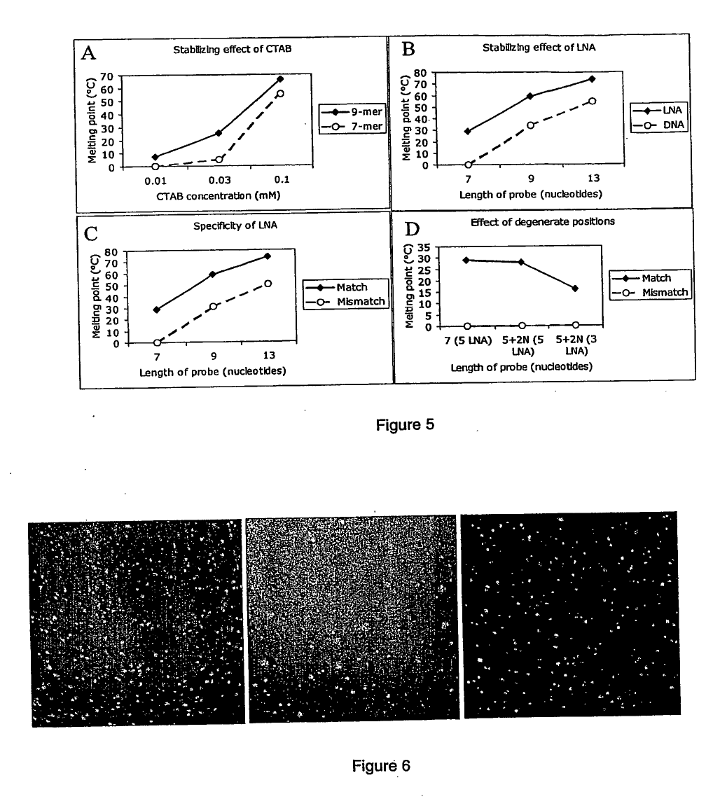

Probes were hybridized in buffer 3 at 100 nM. A temperature ramp was used for each probe to discover the optimal temperature for match/mismatch discrimination. FIG. 5 shows the result of hybridization of two match/mismatch pairs.

1. A nucleic acid sequencing method comprising:
providing a DNA sample containing a plurality of circular single-stranded DNA template molecules each comprising a primer annealing sequence and a target sequence;
forming a random array of immobilized and amplified template molecules, by
contacting said template molecules with an amplification primer to anneal to the primer annealing sequence thereby forming annealed primer/template complexes,
amplifying said template molecules by rolling-circle amplification,
ensuring said amplified template molecules are immobilized on a solid support by immobilizing the amplification primer before annealing the template, the primer/template complexes before amplification, or the amplified templates after amplification;
probing the tandem-repeated amplification product with a panel of probes under test conditions, determining for each probe whether it hybridizes to the target sequences or not under the test conditions, thereby obtaining a hybridization spectrum of the target;
comparing the hybridization spectrum to a hybridization spectrum for reference sequences in a reference database comprising a plurality of reference sequences, wherein the reference database is expected to contain within it one or more reference sequences for the sequence of the DNA template, thereby determining the likely location or locations of the target sequence within one or more reference sequences;
optionally computing the likely sequence of the target sequence and/or a difference in sequence of the target sequence compared with one or more reference sequences by comparing the actual hybridization spectrum with the expected hybridization spectrum at the location or locations.

2. A method according to claim 1 comprising computing a difference in sequence of the target sequence compared with one or more reference sequences, wherein the difference is one or more or a combination of differences selected from the group consisting of single nucleotide polymorphism, insertion, deletion, alternative splicing, an alternative transcriptional start site, alternative polyadenylation, and microsatellites.

3. A method according to claim 1 wherein the panel of probes comprises probes with an effective specificity of 3 to 10 bases.

4. A method according to claim 3 wherein said effective specificity is 4 to 6 bases.

5. A method according to claim 1 wherein the size of each target sequence and the effective specificity of the full or partial panel of probes are adjusted so that the statistical probability of hybridization of each probe to each target is between 5% and 95%.

6. A method according to claim 5 wherein said statistical probability is between 10% and 90%.

7. A method according to claim 6 wherein said statistical probability is between 25% and 75%.

8. A method according to claim 7 wherein said statistical probability is between 40% and 60%.

9. A method according to claim 1 comprising probing with multiple panels of probes, where each probe in each panel of probes is different from each probe in each other panel of probes.

10. A method according to claim 1 wherein the reference database is compiled from sequences of nucleic acid from the same species as the target sequence.

11. A method according to claim 1 wherein the reference database is compiled from sequences of nucleic acid from a different species from the target sequence.

12. A method according to claim 1 comprising forming a random array of single-stranded DNA molecules, wherein
each said molecule consists of at least two tandem-repeated copies of an initial sequence,
each said molecule is immobilized on a surface at random locations with a density of between 10³ and 10⁷ per cm²,
each said initial sequence represents a random fragment from an initial target DNA or RNA library comprising a mixture of single- or double-stranded RNA or DNA molecules,
said initial sequences of all said DNA molecules have approximately the same length.

13. A method according to claim 12 wherein each molecule comprises at least 1000 tandem-repeated copies of an initial sequence.

14. A method according to claim 12 wherein said density is between 10⁵ per cm² and 10⁷ per cm².

15. A method according to claim 12 wherein said initial sequences have the same length within 50% CV.

16. A method according to claim 15 wherein said initial sequences have the same length within 10% CV.

17. A method according to claim 16 wherein said initial sequences have the same length within 5% CV.

18. A method according claim 12 wherein said initial target library is an RNA library, an mRNA library, a cDNA library, a genomic DNA library, a plasmid DNA library or a library of DNA molecules.

19. A method according to claim 1 wherein, in the panel of probes:
each probe consists of one or more oligonucleotides,
each said oligonucleotide is stabilized,
each said oligonucleotide carries a reporter moiety,
the effective specificity of each probe is between 3 and 10 bp,
the set of probes is such that at least 10% of all positions in a random or arbitrary target sequence statistically hybridize with at least one probe in the set of probes.

20. A method according to claim 19 wherein the effective specificity is between 4 and 6 bp.

21. A method according to claim 19 wherein the panel of probes statistically hybridizes to at least 25% of all positions in a target sequence.

22. A method according to claim 21 wherein the panel of probes statistically hybridizes to at least 50% of all positions in a target sequence.

23. A method according to claim 22 wherein the panel of probes statistically hybridizes to at least 90% of all positions in a target sequence.

24. A method according to claim 23 wherein the panel of probes statistically hybridizes to 100% of all positions in a target sequence.

25. A method according to claim 19 stabilised by one or more of introduction of degenerate positions, introduction of locked nucleic acid monomers, introduction of peptide nucleic acid monomers and introduction of a minor groove binder.

26. A method according to claim 19 wherein the reporter moiety is selected from the group consisting of a fluorophor, a quencher, a dark quencher, a redox label, and a chemically reactive group which can be labeled by enzymatic or chemical means, for example a free 3′-OH for primer extension with labeled nucleotides or an amine for chemical labelling after hybridization.

27. A method according to claim 1, wherein the hybridisation spectra are compared using a spectral search instrument comprising a field-programmable gate array (FPGA) attached to a host computer and a computer-readable memory device, wherein
said FPGA is configured to perform spectral search,
said computer-readable memory device stores a reference nucleotide sequence and a set of hybridization spectra,
said host computer is configured to provide said FPGA with the reference nucleotide sequence and with each said hybridization spectrum,
said FPGA, when provided with a reference nucleotide sequence and a hybridization spectrum, writes to said computer-readable memory to store the location or locations of best matches between said hybridization spectrum and said reference nucleotide sequence.

28. A computer processor programmed to control a method of according to claim 1.

29. A computer-readable device carrying a program for a computer processor according to claim 28.

30. A computer processor programmed to provide sequence information for a nucleic acid from performance of a method according to claim 1.

31. A computer-readable device carrying a program for a computer processor according to claim 30.

32. A random array of single-stranded DNA molecules, wherein
each said molecule consists of at least two tandem-repeated copies of an initial sequence,
each said molecule is immobilized on a surface at random locations with a density of between 10³ and 10⁷ per cm²,
each said initial sequence represents a random fragment from an initial target DNA or RNA library comprising a mixture of single- or double-stranded RNA or DNA molecules, said initial sequences of all said DNA molecules have approximately the same length.

33. A random array according to claim 32 wherein each molecule comprises at least 1000 tandem-repeated copies of an initial sequence.

34. A random array according to claim 32 wherein said density is between 105 per cm² and 107 per cm².

35. A random array according to claim 32 wherein said initial sequences have the same length 20 within 50% CV.

36. A random array according to claim 35 wherein said initial sequences have the same length within 10% CV.

37. A random array according to claim 36 wherein said initial sequences have the same length within 5% CV.

38. A random array according to claim 32 wherein said initial target library is an RNA library, an mRNA library, a cDNA library, a genomic DNA library, a plasmid DNA library or a library of DNA molecules.

39. A set of probes wherein
each probe consists of one or more oligonucleotides,
each said oligonucleotide is stabilized,
each said oligonucleotide carries a reporter moiety,
the effective specificity of each probe is between 3 and 10 bp,
the set of probes is such that at least 10% of all positions in a random or arbitrary target sequence statistically hybridize with at least one probe in the set of probes.

40. A set of probes according to claim 39 wherein the effective specificity is between 4 and 6 bp.

41. A set of probes according to claim 39 which statistically hybridizes to at least 25%, at least 50%, at least 90% of all positions in a target sequence.

42. A set of probes according to claim 41 which statistically hybridizes to 100% of all positions in a target sequence.

43. A set of probes according to claim 39 stabilised by one or more of introduction of degenerate positions, introduction of locked nucleic acid monomers, introduction of peptide nucleic acid monomers and introduction of a minor groove binder.

44. A set of probes according to claim 39 wherein the reporter moiety is selected from the group consisting of a fluorophor, a quencher, a dark quencher, a redox label, and a chemically reactive group which can be labeled by enzymatic or chemical means, for example a free 3′-OH for primer extension with labeled nucleotides or an amine for chemical labelling after hybridization.

45. A spectral search instrument comprising a field-programmable gate array (FPGA) attached to a host computer and a computer-readable memory device, wherein
said FPGA is configured to perform spectral search,
said computer-readable memory device stores a reference nucleotide sequence and a set of hybridization spectra,
said host computer is configured to provide said FPGA with the reference nucleotide sequence and with each said hybridization spectrum,
said FPGA, when provided with a reference nucleotide sequence and a hybridization spectrum, writes to said computer-readable memory to store the location or locations of best matches between said hybridization spectrum and said reference nucleotide sequence.
